(19) United States
(12) Patent Application Publication
(10) Pub. No.: US 2017/0105935 A1
(43) Pub. Date: Apr. 20, 2017

(54) PHARMACEUTICAL COMPOSITION CAPABLE OF ACCELERATED PENETRATION INTO AFFECTED AREA

(71) Applicants: NAKASHIMA KOGYO CORPORATION, Gifu-Shi (JP); NATIONAL CENTER FOR GERIATRICS AND GERONTOLOGY, Obu-Shi (JP)

(72) Inventors: Yoji NAKASHIMA, Gifu-Shi (JP); Misako NAKASHIMA, Obu-Shi (JP); Koichiro IOHARA, Obu-Shi (JP); Kazumasa YAMADA, Obu-Shi (JP)

(73) Assignees: NAKASHIMA KOGYO CORPORATION, Gifu-Shi (JP); NATIONAL CENTER FOR GERIATRICS AND GERONTOLOGY, Obu-Shi (JP)

(21) Appl. No.: 15/367,616

(22) Filed: Dec. 2, 2016

Related U.S. Application Data
(63) Continuation of application No. PCT/JP2015/082879, filed on Nov. 24, 2015.

(30) Foreign Application Priority Data
Nov. 24, 2014 (JP) 2014-237241

(51) Int. Cl. A61K 9/08; A61K 31/65; A61K 9/00; A61L 27/38; A61C 19/06; A61L 27/50; A61K 31/155; B01F 17/00; B01J 13/00; A61C 5/50; A61K 9/48; A61L 27/54

(52) U.S. Cl. CPC A61K 9/08; A61K 9/4808; A61K 31/65; A61K 9/0019; A61L 27/3834; A61L 27/3865; A61L 27/54; A61L 27/50; A61K 31/155; B01F 17/0007; B01J 13/0069; A61C 5/50; A61C 19/06; A61L 2430/12; A61L 2300/252; A61L 2300/412; A61L 2400/06; A61L 2300/64

(57) ABSTRACT

Provided is a pharmaceutical composition excellent in capability of an accelerated penetration into an affected area. The pharmaceutical composition capable of accelerated penetration into the affected area is formed by dispersing nano-bubbles, which are negatively charged and are given high internal pressures due to their surface tension, in the pharmaceutical composition in a form of a liquid or a gel including a predetermined drug.

This application is a continuation of the International Application No. PCT/JP2015/082879 filed on Nov. 24, 2015, the entireties of which are incorporated herein by reference.

### BACKGROUND OF THE INVENTION

Field of the Invention

The present invention relates to a pharmaceutical composition capable of accelerated penetration into an affected region, and more particularly to a pharmaceutical composition which allows a drug to effectively penetrate into a target affected region by utilizing nano-sized minute bubbles which have an excellent effect to accelerate the penetration, and further to a drug-delivery technique using the pharmaceutical composition.

“The pharmaceutical composition capable of accelerated penetration into affected region” according to the invention can be selected from various known drug forms, such as a capsule, a liquid, a syrup, an ointment, an eye drop, a suppository, an inhalant, an injection, and a parenteral infusion. The invention reveals a drug-delivery technique for medical drugs and its use, wherein the pharmaceutical composition in the above-mentioned form is introduced into the target affected region using a conventional medical equipment such that the desired drug can effectively penetrate even into a narrow gap or path in the affected region.

Further, the invention is applied to a medical treatment in general. In a dental treatment, for example, a pharmaceutical composition is provided for dental treatment, which composition is introduced into the target portion of a periapical lesion or a dental caries by using a conventional medical equipment such as a syringe and a plate for the dental treatment, such that the desired drug can effectively penetrate even into a narrow gap in the affected region.

Description of Related Art

The inventors searched for various kinds of technical documents in the field of medical treatment in order to obtain prior art in connection with the pharmaceutical composition capable of accelerated penetration into the affected region by using nano-bubbles according to the present invention. However, they did not find any document relating directly to the present invention, and found only a medical technique using an ultrasonic wave, for example, as a means for accelerating penetration of conventional drugs into affected regions. For this reason, the background art of the present invention is described below in view of the actual situation in the field of dental treatment.

First of all, in the dental therapy, the dental caries is one of the two major dental diseases the other of which is a periodontal disease, and about a half of tooth loss is due to the dental caries. Like a cold, the dental caries is commonly seen in any generation. However, teeth have low degrees of calcification for several years after eruption, and are especially vulnerable to the dental caries. Thus, the dental caries is often seen in persons underage (minor). A dental pulp, a so-called nerve of a tooth, has functions of blocking an external stimulus by tooth (dentin) formation potential and inhibiting development of the dental caries and tooth fracture by sense. The dental pulp also significantly contributes to tooth survival by metabolic and immune systems.

With a current pulpectomy technique, complete pulpectomy and filling of a root canal are almost impossible, and abnormality (periapical periodontitis) occurs in apical areas in a later stage, leading to the necessity of treatment of an infected root canal in many cases. In such cases, a single chair time is longer than that in a dental caries treatment, and a long-term treatment is often needed. It is also possible that symptoms such as drainage from the apical foramen or a tooth pain are not improved, resulting in a tooth loss by tooth extraction. The tooth pain causes severe difficulties in daily life, reducing social productivity. As the dental caries grows to reach the dental pulp and further the bone in the apical area to finally cause the tooth extraction, mental and economic burdens of on the patients increase, and deterioration of oral and occlusal functions causes motor dysfunction, autonomic imbalance, and problems in pronunciation and aesthetics.

In conventional dental caries treatments, it is necessary to physically completely remove the dental caries (softened dentin) in order to completely kill bacteria which have invaded deep parts of the dentin tubules. This removal increases the possibility of an excessive loss of the dentin, leading to exposure of the dental pulp.

There is also a conventional root canal treatment wherein a portion suffering from the dental caries is removed, and the inside of the root canal are cleaned and disinfected. In the root canal treatment, the portion suffering from the dental caries is first removed, and then the enamel and dentin are removed as necessary in order to easily perform the root canal treatment. Next, after the depth of the root canal has been precisely measured with the sense of fingers, X-ray photographs, or an electric root canal length measurement, for example, the dental pulp or dentin infected by bacteria is removed with a tool such as a reamer or a file. Thereafter, a predetermined drug is put in the root canal from which the dental pulp has been removed, and the root canal is irrigated, cleaned, and disinfected with a tool such as a broach. Lastly, the root canal is filled with a gutta-percha, thus finishing the root canal treatment.

The root canal treatment is classified into the pulpectomy treatment and the infected root canal treatment. If the dental caries is deep enough to reach the dental pulp, the pulpectomy treatment is generally employed. The pulpectomy treatment is a removal of the dental pulp inside the tooth. The dental pulp which is or might be infected is thoroughly removed by the pulpectomy treatment. Thus, it is possible to prevent expansion of inflammation into the periodontal tissue, and the tooth affected by the caries is made harmless to periodontal tissue, whereby the chewing ability is recovered.

An infected root canal refers to a state in which the dental caries develops to the dental pulp and causes necrosis of the dental pulp due to infection, or a state in which insufficient filling of the root canal causes infection of the inside of the root canal. A treatment employed in these cases is called the infected root canal treatment. In a severely infected root canal, an apical periodontal cyst or a fistel (a passageway through which a pus accumulated around the tooth is drained) is created. In the case where the root canal is infected, it is necessary to clean and disinfect the inside of the root canal before filling the root canal. If the root canal was insufficiently filled with a root canal filler in the past, the root canal filler is temporarily removed so that the inside of the root canal is cleaned and disinfected again, and then the root canal is filled to the root apex.

However, it is difficult to directly observe the structure in the root canal, and the shape of the root canal is complex such that the root canal is curved or blocked and has a large number of accessory canals, lateral branches, or the like. Thus, it is very difficult to remove bacteria completely in the root canal. In addition, if the root canal is filled with a filler or covered with a crown, with bacteria being left in the root canal, the bacteria will proliferate in the root canal later in some cases, leading to the necessity of an additional root canal treatment. In a case where the additional root canal treatment is required later, the filling or the crown used in the previous treatment needs to be replaced with a new one, and further, the tooth extraction may be required. If the treatment is excessively focused on removal of residual bacteria in the root canal, the dentin may be excessively removed so as to cause a tooth fracture, or even the tooth itself may be removed, resulting in deterioration of the patients' quality of life (QOL). Since the shape of the root canal is complex as described above, the root canal treatment (treatment of the dental nerves and roots) is quite difficult.

Meanwhile, JP-A-2009-045455 discloses a system for irrigation of the tooth root canal using an ultrasonic energy. The system for irrigation of the tooth root canal includes an injection tube having a flexible distal end configured to be inserted into the root canal. This injection tube is inserted into the root canal so that a fluid having the ultrasonic energy superimposed thereon is forced into the root canal, thus performing the irrigation.

In the system for irrigation of the tooth root canal described in Patent Document 1, however, the fluid having the ultrasonic energy superimposed thereon is merely released to the apical area of the root canal, so that it is difficult to irrigate minute portions of the root canal with a complex shape.

JP-A-2007-229110 discloses a system for the tooth root canal treatment, which system includes a motor for rotatably driving a root canal drill. Driving of this motor is controlled in the following manner. When the root canal drill is inserted into the root canal with the motor rotated in the direction opposite to the rotating direction of the root canal drill for cutting the root canal, rotation in the opposite direction of the root canal drill is maintained until an electric root-canal-length measuring means detects that the distal end of the root canal drill reaches a predetermined reference position. When the electric root-canal-length measuring means detects that the distal end of the root canal drill has reached the reference position, the rotation in the opposite direction of the root canal drill is stopped.

In the system for the tooth root canal treatment described in Patent Document 2, the root canal drill can be rotated in the direction opposite to the direction of rotation for cutting the root canal. In this system, after the root canal has been drilled and enlarged by rotating the root canal drill in the positive direction, a drug solution is injected into the root canal. Then, the root canal drill is rotated in the opposite direction and inserted into the root canal, and then e root canal drill is rotated in the positive direction so that produced chips are ejected to the proximal end (upstream) of the root canal drill. Accordingly, the above-mentioned insertion of the drill with the rotation in the opposite direction pushes the drug solution toward the distal end of the root canal drill.

In the system for the tooth root canal treatment described in Patent Document 2, however, the root canal drill might excessively drill and enlarge the root canal. In addition, although the drug solution is sufficiently injected into the distal end of the root canal drill, the lateral branches and accessory canals of the root canal are not sufficiently taken into consideration, so that it is difficult to irrigate minute portions of the complex root canal.

JP-A-2004-313659 describes a dental therapeutic system in which a liquid supply nozzle for supplying a drug solution (a therapeutic solution) or the like and a suction nozzle are inserted into the root canal with their tips being positioned at different locations, and a drug solution is injected so that the drug solution fully penetrates into the root canal. If the liquid supply nozzle and the suction nozzle are positioned such that one of these nozzles is located at a portion deeper than the other in the cavity, the treatment solution reaches at least the deep portion in the cavity. Accordingly, a target region is efficiently irrigated.

In the dental therapeutic system described in Patent Document 3, however, the apical area can be irrigated, but the irrigation of the other areas are insufficient, since the tip apertures of the liquid supply nozzle and the suction nozzle for the drug solution face the apical area of the root canal.

The periodontal disease (periodontal disorder) is inflammation of the periodontal tissue which supports the teeth. The periodontal tissue is a general term including the cementum, gingiva, alveolar bone, and periodontium. The periodontal disease is a disease caused by infection with periodontal disease bacteria coming from the so-called gingival sulci (periodontal pockets) between the teeth and the gingivae. The periodontal disease is broadly classified into gingivitis with no alveolar bone resorption, and periodontitis with the alveolar bone resorption. In both of these cases, induced inflammation tends to enlarge the periodontal pockets.

Oral rinses, dentifrices, and antibiotics, for example, are known as conventional therapeutic drugs for the periodontal diseases. Conventional therapeutic methods include brushing with tooth brushes, and dental calculus removal and irrigation performed in dental clinics. However, the use of the dentifrices has the possibility of insufficient cleaning of the periodontal pockets if the brushing is insufficient. In the case of using the oral rinses, although the oral rinses spread in the mouth, the drug solution is not effective in some areas such as the periodontal pockets. In the case of the antibiotics, arrival of medicinal ingredients at inflammated regions such as the gingivae takes too much time after administration, and the antibiotics are not effective against all of the periodontal disease bacteria. JP-A-H11-240816 proposes an embrocation liquid for the periodontal diseases using a shellac as a base material in a tooth coating composition. However, this embrocation liquid has a problem that it cannot be used in the periodontal pockets.

Furthermore, hyperesthesia is a disease in which an advanced periodontal disease causes a transient pain when the surface of exposed dentin is subjected to cold air, cold water, or tactile stimuli, for example. The exposure of the dentin is caused by enamel disappearing or gingival retraction, for example. In the exposed dentin, mechanical wearing or elution of lime due to an effect of an acid or the like forms openings in dentin tubules through which physicochemical stimuli are transmitted to the dental pulp to stimulate sensory nerves and cause a pain.

For hyperesthesia therapy, there is a technique of closing the openings in the dentin tubules. For example, JP-A-H05-155745 describes a technique in which a tooth is subjected to a treatment using a water-soluble aluminum compound and a fluoride. JP-A-H05-155746 shows a technique in which a tooth is subjected to a treatment using a water-soluble aluminum compound, a fluoride, and a water-soluble calcium compound. However, these techniques have a problem that drugs do not easily penetrate into the dentin tubules, so that the closure of the dentin tubules is insufficient.

As described above, the inside of the root canal has a complex shape, and thus, the treatment of the root canal is very difficult to conduct. However, without an appropriate root canal treatment, periapical periodontitis will arise later, resulting in suppuration of the apical area of the root. It is also difficult for the drugs to penetrate into the periodontal pockets and dentin tubules, and adequate treatments for the periodontal disease and hyperesthesia are needed.

### SUMMARY OF THE INVENTION

The present invention was made in view of the background art described above. It is therefore a problem to be solved by the invention to provide a pharmaceutical composition excellent in accelerating penetration of a drug into an affected region. It is another problem to be solved by the invention to provide a pharmaceutical composition for medical care use which allows a prescribed drug to effectively penetrate into the target affected region and advantageously exhibit its pharmacological effect utilizing nano-sized minute bubbles which have an excellent effect of accelerating the penetration of the drug. It is a further problem to be solved by the present invention to provide a pharmaceutical composition excellent in accelerating the penetration of a drug into an affected region for dental care use, which pharmaceutical composition is capable of adequately cleaning the inside of the root canals with complex shapes, and also capable of closing the openings in the dentin tubules and killing bacteria in the dentin tubules by allowing a drug to effectively penetrate into the dentin tubules.

To solve the problems described above, the present invention can be suitably carried out in various modes described below. It is to be understood that the invention may be embodied in any desired combination of those modes, and that the modes and technical features of this invention are not limited to those described below, and should be recognized on the basis of the concept of the invention as disclosed in the specification in its entirety and the drawings.

(1) A pharmaceutical composition in a form of a liquid or a gel capable of accelerated penetration into an affected region, comprising a predetermined drug and containing minute bubbles dispersed therein, characterized in that the minute bubbles are nano-bubbles each of which has a nano-meter diameter and is given a high internal pressure due to its surface tension and negatively charged.
(2) The pharmaceutical composition capable of accelerated penetration into an affected region according to the mode (1), wherein each of the nano-bubbles has a diameter within a range of 10 nm-1000 nm.
(3) The pharmaceutical composition capable of accelerated penetration into an affected region according to the mode (1) or (2), wherein the nano-bubbles are contained and dispersed at a ratio of 1×10⁶-2×10⁸ bubbles/ml.
(4) The pharmaceutical composition capable of accelerated penetration into an affected region according to any one of the modes (1) to (3), wherein the pharmaceutical composition is a mixture obtained by mixing a solution containing the nano-bubbles, and the drug or a liquid or gel-type preliminary composition containing the drug.
(5) The pharmaceutical composition capable of accelerated penetration into an affected region according to any one of the modes (1) to (4), wherein the minute bubbles having the nano-meter diameter are generated by a nano-bubble generating device comprising: a cylindrical gas-permeable portion having a gas-permeable film formed by generating crazes in a high molecular resin film arranged on its gas-permeable cylindrical outer circumferential surface, wherein a predetermined pressurized gas is ejected under the control of an amount of permeation of the gas through the gas-permeable film; gas blowing means for blowing the pressurized gas into an inside of the cylindrical gas-permeable portion; a cylindrical casing which has an inside diameter larger than the outside diameter of the cylindrical gas-permeable portion and which is open at its opposite ends; and fluid flowing means for permitting a predetermined fluid to flow through a fluid passage provided by a gap formed by accommodating the cylindrical gas-permeable portion within the cylindrical casing, and wherein bubbles formed by the gas ejected from the gas-permeable outer circumferential surface of the gas-permeable portion are sheared and micronized by the fluid flowing through the fluid passage at an early stage of generation, whereby the minute bubbles with the nano-meter diameter are formed.
(6) The pharmaceutical composition capable of accelerated penetration into an affected region according to any one of the modes (1) to (5), wherein the pharmaceutical composition is formed as an administrative agent included within a soluble outer shell of a capsule and applied to the affected region.
(7) The pharmaceutical composition capable of accelerated penetration into an affected region according to any one of the modes (1) to (6), wherein the pharmaceutical composition is atomized by spraying, subjected to absorption to a fibrous absorbent such as a non-woven fabric, a woven fabric, a fabric or a Japanese paper, and applied to the affected region.
(8) The pharmaceutical composition capable of accelerated penetration into an affected region according to any one of the modes (1) to (6), wherein the pharmaceutical composition is atomized by spraying and applied directly to the affected region.
(9) The pharmaceutical composition capable of accelerated penetration into an affected region according to any one of the modes (1) to (8), wherein the pharmaceutical composition is intended for medical care use and the drug is subjected to penetration into a deep part of the target region affected by a tooth decay and the like by utilizing an effect of accelerated penetration achieved by the nano-bubbles.
(10) The pharmaceutical composition capable of accelerated penetration into an affected region according to any one of the modes (1) to (9), wherein the pharmaceutical composition is used for dental therapy in the case of a damage or a partial loss of the dental pulp, the pharmaceutical composition being administered in the case of the damage or the partial loss of the dental pulp and permitting formation of the dental pulp and/or the dentin by causing differentiation of the odontoblast from the dental pulp cell at a portion to which the pharmaceutical composition is administered, and wherein the drug includes as an effective ingredient at least one of MMPs, BMPs, bFGF, G-CSF, CXCL14, MCP1, SDF-1, PDGF, GM-CSF, HGF, BDNF and NPY.
(11) The pharmaceutical composition capable of accelerated penetration into an affected region according to any one of the modes (1) to (9), wherein the pharmaceutical composition is used for dental therapy to promote sterilization, anti-inflammation and analgesia or regeneration of the dentin, the dental pulp or the periodontal tissue, the pharmaceutical composition containing as the drug at least one of sodium hypochlorite, hydrogen peroxide, formalin cresol, formalin guaiacol, phenol, phenol camphor, parachlorophenol camphor, cresatin, guaiacol, cresol, iodine tincture, an EDTA product, calcium hydroxide, tetracycline hydrochloride, ampicillin, imipenem, panipenem, vancomycin, chloramphenicol, PBSS, PBSC, ofloxacin, levofloxacin, metronidazole, cefaclor, ciprofloxacin, minocycline, imidazole, a cathepsin K inhibitor, BMPs, bFGF, G-CSF, CXCL14, MCP1, SDF-1, PDGF, GM-CSF, HGF, BDNF and NPY.
(12) The pharmaceutical composition capable of accelerated penetration into an affected region according to any one of the modes (1) to (9), wherein the pharmaceutical composition is used for treating periodontal diseases in dental therapy to promote sterilization, anti-inflammation and analgesia or regeneration of the periodontal tissue, the pharmaceutical composition containing as the drug at least one of iodine tincture, an EDTA product, tetracycline hydrochloride, ampicillin, imipenem, panipenem, vancomycin, chloramphenicol, PBSS, PBSC, ofloxacin, levofloxacin, metronidazole, cefaclor, ciprofloxacin, minocycline, imidazole, a cathepsin K inhibitor, BMPs, bFGF, G-CSF, CXCL14, MCP1, SDF-1, PDGF, GM-CSF, HGF, BDNF, NPY and Emdogain®.
(13) The pharmaceutical composition capable of accelerated penetration into an affected region according to any one of the modes (1) to (9), wherein the pharmaceutical composition is used for curing hyperesthesia in dental therapy, the pharmaceutical composition containing as the drug at least one of potassium nitrate, oxalic acid, a diamine silver fluoride product, copal resin, sodium fluoride, zinc chloride, a water-soluble aluminium compound, a water-soluble calcium compound, BMPs and bFGF.
(14) The pharmaceutical composition capable of accelerated penetration into an affected region according to any one of the modes (1) to (9), wherein the pharmaceutical composition is used for an oral and implant care in dental therapy to promote sterilization, anti-inflammation and analgesia, recalcification of a tooth, or regeneration of the periodontal tissue, the pharmaceutical composition containing as the drug at least one of benzalkonium, chlorhexidine gluconate, sodium N-lauroylsarcosinate, isopropylmethyl phenol, ε-aminocaproic acid, sodium hypochlorite, hydrogen peroxide, formalin cresol, formalin guaiacol, phenol, phenol camphor, parachlorophenol camphor, cresatin, guaiacol, cresol, iodine tincture, an EDTA product, calcium hydroxide, tetracycline hydrochloride, ampicillin, imipenem, panipenem, vancomycin, chloramphenicol, PBSS, PBSC, ofloxacin, levofloxacin, metronidazole, cefaclor, ciprofloxacin, minocycline, imidazole, a cathepsin K inhibitor, BMPs, bFGF, G-CSF, CXCL14, MCP1, SDF-1, PDGF, GM-CSF, HGF, BDNF and NPY.
(15) The pharmaceutical composition capable of accelerated penetration into an affected region according to any one of the modes (1) to (9), wherein the pharmaceutical composition is used in the field of dental or medical therapy via a mucous membrane or a skin, for the purposes of sterilization, disinfection, anti-inflammation and analgesia, protection of the mucous membrane and the skin, and for other purposes.
(16) A method of producing the pharmaceutical composition capable of accelerated penetration into an affected region according to the mode (1), comprising: a step of providing a drug to be penetrated into a target affected region or a liquid or gel-type preliminary composition containing the drug; a step of preparing a solution containing the nano-bubbles by using a nano-bubble generating device comprising: a cylindrical gas-permeable portion having a gas-permeable film formed by generating crazes in a high molecular resin film arranged on its gas-permeable cylindrical outer circumferential surface, wherein a predetermined pressurized gas is ejected under the control of an amount of permeation of the gas through the gas-permeable film; gas blowing means for blowing the pressurized gas into an inside of the cylindrical gas-permeable portion; a cylindrical casing which has an inside diameter larger than the outside diameter of the cylindrical gas-permeable portion and which is open at its opposite ends; and liquid flowing means for permitting a predetermined liquid to flow through a liquid passage provided by a gap formed by accommodating the cylindrical gas-permeable portion within the cylindrical casing, the device being characterized in that bubbles formed by the gas ejected from the gas-permeable outer circumferential surface of the gas-permeable portion are sheared and micronized by the liquid flowing through the liquid passage at an early stage of generation, whereby the minute bubbles with the nano-meter diameter are formed; and a step of mixing the obtained solution containing the nano-bubbles, and the drug or the liquid or gel-type preliminary composition containing the drug so as to obtain the pharmaceutical composition.
(17) The method of producing the pharmaceutical composition capable of accelerated penetration into an affected region according to the mode (16), wherein the amount of inclusion of the nano-bubbles in a nano-bubble-containing liquid is increased by repeatedly reintroducing the nano-bubble-containing liquid flowing from an outlet opening of the casing into the nano-bubble generating device so that the nano-bubbles are further generated through the gas-permeable member in the nano-bubble-containing liquid, whereby the liquid having a desired concentration of the nano-bubbles can be obtained, so that the pharmaceutical composition containing a predetermined size of the nano-bubbles within a predetermined range of concentration can be obtained.

As described above, the pharmaceutical composition for medical care use in the form of a gel or a liquid according to the present invention contains nano-sized minute bubbles (hereinafter referred to as “nano-bubbles”). The nano-bubbles have excellent surface characteristics and effective motion characteristics such as the Brownian motion, so that also the drug is attracted and directed by those bubbles for effective entry or penetration into a target affected region, such as the inside of a complex structure of the root canal, whereby the inside of the root canal is adequately subjected to an operation or treatment of cleaning, sterilization, disinfection and the like. Furthermore, in the field of dental therapy, the drug penetrates into small gaps of the periodontal pockets, so as to permit adequate treatments such as the cleaning, sterilization and disinfection with respect to the inside of the periodontal pockets, cementum or dentin. Even hyperesthesia can be adequately treated by penetration of the drug into the dentin tubules.

It is further noted that the use of the pharmaceutical composition according to the present invention especially in the dental therapy permits quick and accurate sterilization of the bacteria within the root canal and the dentin tubules, so as to advantageously prevent an excessive removal of the dentin, and fracture and removal of teeth. As a result, the frequency of visit of a patient to a clinic and the length of time required for treatment of the patient can be effectively reduced, whereby a high-quality and efficient dental therapy can be realized.

### BRIEF DESCRIPTION OF DRAWINGS

FIG. 1 is a schematic cross sectional view showing one example of a bubble generating device which is capable of advantageously generating desired nano-bubbles within a fluid.

FIGS. 2A-2E are pictures showing a result of a disinfection test in vitro of the root canal using an extracted canine front tooth, which test is described in Experiment 1.

FIG. 2(A) are pictures showing generation of fluorescence all over the root canal wall (an experimental control) in treatment A), FIG. 2 (B) are pictures showing generation of fluorescence in a deep part not less than 600-700 μm from the root canal wall in treatment B), and FIG. 2 (C) are pictures showing generation of fluorescence in a deep part not less than 900-1000 μm from the root canal wall in treatment C). FIG. 2 (D) are pictures showing generation of fluorescence in a deep part not less than 1000-1300 μm from the root canal wall in treatment D), and FIG. 2 (E) are pictures showing generation of fluorescence in a deep part not less than 1000-1500 μm from the root canal wall in treatment E).

FIG. 3 is a graph showing a result of a disinfection test in vivo of a tooth having an infected root canal using nano-bubbles and an antibacterial drug, which test is described in Experiment 2.

FIGS. 4A and 4B are pictures showing a result of a test in vivo of regeneration of the dental pulp and periapical tissue of a tooth having an infected root canal using the nano-bubbles and the antibacterial drug, which test is described in Experiment 3. FIG. 4 (A) is a picture showing the case of cell transplantation after application of the nano-bubbles, and FIG. 4 (B) is a picture showing the case of cell transplantation without application of the nano-bubbles.

FIG. 5 is a graph showing an in vivo effect of a decrease of the number of bacteria within the periodontal pocket by applying nano-bubbles and an antibacterial disinfecting drug to a plate in Experiment 5.

FIG. 6 is a picture showing a result of an examination of nano-bubbles with respect to safety in vivo in Experiment 6.

### DETAILED DESCRIPTION OF THE INVENTION

To further clarify the structure of the pharmaceutical composition capable of accelerated penetration into an affected region according to the present invention, embodiments of the present invention will be described in detail by reference to the drawings.

The pharmaceutical composition capable of accelerated penetration into the affected region according to the present invention is in the form of a liquid or a gel containing minute or ultrafine bubbles, which are introduced therein with nano-meter diameters so as to be given a high internal pressure due to their surface tension and negatively charged. In other words, the minute or ultrafine bubbles are nano-bubbles having a high internal pressure and an electric charge. The surface characteristics and the motion characteristics such as the Brownian motion of the nano-bubbles permit the nano-bubbles to have an effective function of the accelerated penetration, resulting in the characteristic of the nano-bubbles having the capability of advantageously introducing the target drug into the deep part of the affected region without inducting the bubbles by an ultrasonic device or the like. It is noted that, in the pharmaceutical composition in the form of a liquid or a gel according to the present invention, the drug may be dissolved in the composition, or may take the form of fine particles. The fine particles may be, for example, contained in a dispersed form, wherein the particles of the drug having diameters of about 0.001 μm-10 μm are in the form of floatage or suspension in the composition.

In view of ease of delivery into the complicated structure of the affected region like the root canal and the tissue in the deep part, and further in view of stability of the nano-bubbles, the size of the nano-bubbles included in the pharmaceutical composition is, preferably within the range of 10-1000 nm, especially 10-800 nm. More preferably, the size is within the range of 10-500 nm, and further preferably 100-400 nm. By setting the diameter (bubble diameter) of the nano-bubbles within the range described above, the nano-bubbles are advantageously delivered to the affected region such as the root canal, lateral branches, dentin tubules and the like. In the case where the size of the nano-bubbles is excessively large, the capability of penetration of the drug into the affected region is deteriorated.

Furthermore, the nano-bubbles in the above-described pharmaceutical composition have a high internal pressure due to their surface tension, and are negatively charged, because of their nano-sized minute diameters. The internal pressure of the nano-bubbles generally corresponds to the diameter of the nano-bubbles and is calculated according to the Young-Laplace formula. It is considered that the nano-bubbles according to the present invention have an internal pressure of about 3-300 atmospheric pressure. Furthermore, a zeta potential of the nano-bubbles in the water is assumed to be from −30 mV to −40 mV in general, meaning that the nano-bubbles are negatively charged. It is considered that this electric charge property permits the nano-bubbles to catch and introduce the drug components into the deep part of the affected region.

The amount of existence of the nano-bubbles in the pharmaceutical composition is generally represented by the number of the bubbles in a predetermined volume of the pharmaceutical composition. In the present invention, the nano-bubbles are advantageously dispersed and contained at a ratio of 1×10⁶-2×10⁸/ml, preferably at a ratio of 5×10⁶-1.5×10⁸/ml, and more preferably at a ratio of 1×10⁷-1×10⁸/ml. In the case where the amount of existence of the nano-bubbles is excessively small, the effective function of the accelerated penetration achieved by the nano-bubbles being negatively charged and having a high internal pressure cannot be achieved. On the other hand, in the case where the amount of existence of the nano-bubbles is excessively large, the function of the accelerated penetration tends to be saturated, with a limited economical advantage.

The size and amount of existence of the above-described nano-bubbles can be measured with commercially available nano-particle analyzers such as a nano-particle distribution analyzer available from Shimadzu Corporation (SALD-7100) and a nano-particle analyzer available from Quantum Design Japan, Inc. (NanoSight LM-20), for example.

The pharmaceutical composition containing the nano-bubbles according to the present invention can be formed by directly introducing the desired nano-bubbles into the predetermined pharmaceutical composition (a drug per se or a preliminary composition including the drug). However, especially preferably, the pharmaceutical composition is formed by mixing a liquid such as water containing the nano-bubbles and a drug or a preliminary composition in the form of a liquid or a gel including the drug, so as to permit easy and advantageous introduction of the desired nano-bubbles into the pharmaceutical composition. That is, a liquid containing the desired nano-bubbles is prepared in advance and uniformly mixed into the predetermined drug or its preliminary composition, so that various kinds of the desired pharmaceutical composition can be easily obtained.

In the present invention, the nano-bubbles having nano-sized diameters which are introduced into the pharmaceutical composition can be formed by using various known nano-bubble generating devices. In particular, a device having a gas-permeable film formed by generating crazes in a high molecular resin film is advantageously used. The gas-permeable film permits release of a predetermined gas under control of the amount of gas permeation so as to form the nano-bubbles. Such a device is disclosed in Japanese Patent Nos. 3806008 and 5390212, for example.

Among the known devices, in the present invention, a nano-bubble generating device comprising the following is preferably selected: a cylindrical gas-permeable portion having a gas-permeable film formed by generating crazes in a high molecular resin film arranged on its gas-permeable cylindrical outer circumferential surface, wherein a predetermined pressurized gas is ejected under the control of an amount of permeation of the gas through the gas-permeable film; gas blowing means for blowing the pressurized gas into the cylinder of the cylindrical gas-permeable portion; a cylindrical casing which has an inside diameter larger than the outside diameter of the cylindrical gas-permeable portion and which is open at its opposite ends; and fluid flowing means for permitting a predetermined fluid to flow through a fluid passage provided by a gap formed by accommodating the cylindrical gas-permeable portion within the cylindrical casing. In the above-described device, bubbles formed by the gas ejected from the gas-permeable outer circumferential surface of the gas-permeable portion are sheared and micronized by the fluid flowing through the fluid passage at an early stage of generation, whereby the minute bubbles (nano-bubbles) having the nano-meter diameters, high internal pressures and negative charges, are effectively formed.

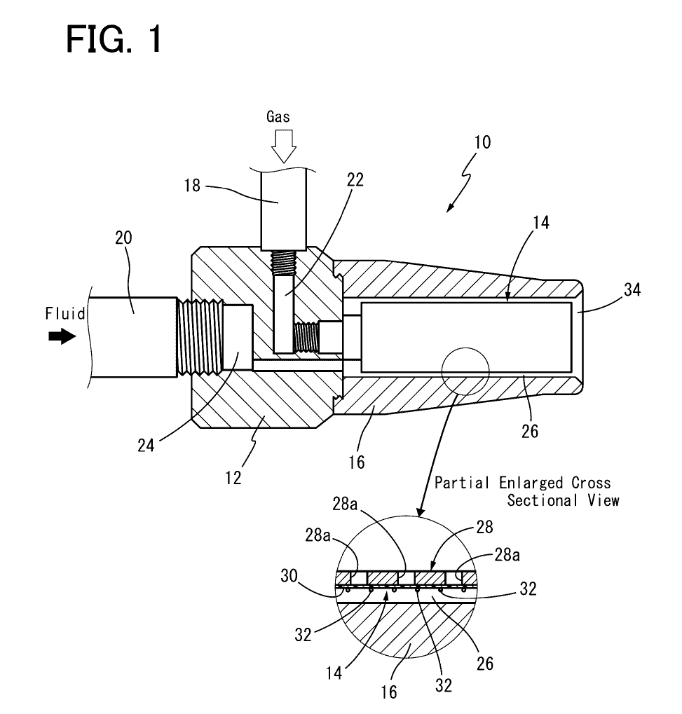

Referring to the cross sectional view of FIG. 1, there is schematically shown one embodiment of the above-described nano-bubble generating device. The nano-bubble generating device is constituted so as to include: a base (base stand) 12; a cylindrical gas-permeable member 14 concentrically attached to the base 12; a cylindrical casing 16; a gas-introducing tube 18 through which a predetermined gas such as air is provided under pressurization; and a fluid-introducing tube 20 through which a fluid (liquid) such as water is introduced.

In the nano-bubble generating device 10, a gas-introducing passage 22 and a fluid-introducing passage 24 are provided within the base 12 independently of each other. The gas-introducing tube 18 is attached to one open end portion of the gas-introducing passage 22 in the base 12, while to the other side of open end portion of the gas-introducing passage 22, a proximal end portion of the gas-permeable member 14 is gastightly attached. The fluid-introducing tube 20 is attached to one open end portion of the fluid-introducing passage 24 in the base 12, while the casing 16 is attached to the base 12 concentrically with the gas-permeable member 14 such that the other open end of the fluid-introducing passage 24 is open to the cylindrical inside of the casing 16.

The casing 16 has an inside diameter larger than an outside diameter of the cylindrical gas-permeable member 14, and is in the form of the cylinder open at its opposite ends, so that when one end of the casing 16 is fixed to a predetermined fixing part of the base 12, a difference between the outside diameter of the cylindrical gas-permeable member 14 and the inside diameter of the cylindrical casing 16 causes a predetermined gap to be formed between the outer circumferential surface of the gas-permeable member 14 (gas-permeable surface) and the inner circumferential surface of the casing 16. This gap provides a fluid passage 26.

As shown in the cross sectional view of FIG. 1, the gas-permeable member 14 is constituted by a cylindrical member 28, and a gas-permeable film (crazed film) 30 on the outer circumferential surface of the cylindrical member 28 formed by generating crazes on a high molecular resin film. The crazed film 30 has a binding string 32 on its outer circumferential surface, so as to be fixedly held on the outer circumferential surface of the cylindrical member 28. The cylindrical member 28 has a proximal open end and a distal closed end. To the opening of the proximal open end of the cylindrical member 28, the above-described other open end of the gas-introducing passage 22 is fixed for communication with the cylindrical member 28.

The crazed film 30 constituting the gas-permeable member 14 is a gas-permeable film obtained by subjecting a high molecular resin film to a conventional crazing treatment to generate crazes and thereby giving the resin film gas permeability, as disclosed in Japanese Patent Nos. 3156058 and 5390212. Generally, the crazed film exhibits a water repellent property, and has a known structure having a multiplicity of minute pores which permit permeation of a gas but do not permit permeation of water or any other liquid, and a gel solution.

The high molecular resin used for the crazed film 30 may be selected from polyolefin, polyester, polyamide, styrene resins, polycarbonate, halogen-contained thermosetting resins, and nitrile resins, for example. Specific examples of the above-indicated resins may be those as disclosed in Japanese Patent No. 3806008, for example. The crazed film 30 is formed from one of those resin materials or a combination of two or more of the resin materials. It is noted that the crazed film 30 may consist of a single layer or a plurality of layers superposed on each other.

The thickness of the crazed film 30 is not particularly limited, but is generally selected within a range of 0.5-1000 μm, preferably within a range of 1-800 μm, and more preferably within a range of 2-500 μm. The crazes formed in the crazed film 30 basically take the form of stripes extending almost parallel to the direction of molecular orientation of the high molecular resin film, each stripe having a width within a range of 0.5-100 μm, preferably within a range of 1-50 μm. A ratio of the number of crazes in the form of stripes formed through the entire thickness of the film in terms of the total number of the crazes is preferably at least 10%, more preferably at least 20%, and further preferably at least 40%. Where the percentage of the number of the crazes formed through the film is lower than the above-indicated lower limit, the crazed film is less likely to assure a sufficient degree of gas permeability. The other properties of the crazed film 30, other structural features of the crazes, and the method of production of the crazes are similar to those described in Japanese Patent No. 3806008.

When the desired nano-bubbles are generated by using the nano-bubble generating device 10, a liquid such as a pressurized water the pressure of which is regulated and which is delivered by a delivering device like a delivering pump is passed through the fluid-introducing tube 20 and the fluid-introducing passage 24, and introduced into the fluid passage 26 formed between the outer circumferential surface (gas-permeable surface) of the gas-permeable member 14 and the inner circumferential surface of the cylindrical casing 16. Meanwhile, a gas such as a compressed air the pressure of which is regulated by a compressor (not shown) or a gas such as a pressurized air the pressure of which is regulated and which is delivered by a pressure-resistant cylinder (not shown) or the like is passed through the gas-introducing tube 18 and the gas-introducing passage 22, and introduced into the inside of the cylindrical member 28 of the gas-permeable member 14, by way of the open end portion of the gas-permeable member 14. Then, through multiple gas inlet holes 28a formed on the cylindrical member 28 of the gas-permeable member 14, the pressurized gas permeates through the crazes in the crazed film 30 placed on the outer circumferential surface of the gas-permeable member 14, which crazed film 30 can regulate the amount of permeation of the gas. The pressurized gas is then ejected into the fluid passage 26 so as to form the bubbles. The bubbles are subjected to shearing and micronization at the early stage of formation by a flow of the fluid passing through the fluid passage 26, whereby the nano-bubbles are generated. The gas introduced through the gas-introducing tube 18 is generally an air. However, it is noted that any known gas such as nitrogen, argon and helium can be used as long as the gas does not dissolve in or react with the fluid introduced through the fluid-introducing tube 20.

In the case where the gas is the air, the nano-bubble generating device 10 permits easy introduction of the nano-bubbles with a size of 10 nm-5 μm, specifically not larger than 1000 nm, more specifically not larger than 500 nm in general, into the fluid like water passed through the fluid passage 26. Among the generated minute bubbles, large bubbles which can be seen by the eyes come to the surface of the fluid and disappear. On the other hand, minute nano-bubbles with a size of not larger than 1000 nm, which are difficult to be seen by the eyes, have a low degree of buoyancy, so that the nano-bubbles stay in the fluid, with a sort of motion like the Brownian motion, whereby the fluid containing a large amount of the nano-sized minute bubbles can be advantageously obtained. In addition, the nano-bubbles obtained as described above are given a high internal pressure due to their surface tension and are negatively charged.

In the nano-bubble generating device 10, the liquid containing the predetermined nano-bubbles can be advantageously obtained by using a liquid such as water, a solvent and various kinds of solution as a fluid introduced through the fluid-introducing tube 20. By mixing such liquid containing the nano-bubbles with the predetermined drug or the preliminary composition in the form of a liquid or a gel containing the drug, the pharmaceutical composition capable of accelerated penetration into the affected region according to the present invention is easily obtained. In the case where the fluid introduced through the fluid-introducing tube 20 is a liquid containing a predetermined drug dissolved or dispersed therein, the nano-bubbles are generated directly in the drug-containing liquid so that the desired pharmaceutical composition capable of accelerated penetration into the affected region can be obtained directly. Furthermore, the fluid introduced through the fluid-introducing tube 20 can be a predetermined drug or a preliminary composition in the form of a gel containing the predetermined drug. In this case too, the desired pharmaceutical composition capable of accelerated penetration into the affected region can be obtained.

Meanwhile, the amount of inclusion of the nano-bubbles in the nano-bubble-containing liquid or gel composition obtained as described above can be increased by repeatedly reintroducing the nano-bubble-containing liquid or gel composition flowing from an outlet portion of the nano-bubble-containing fluid in the nano-bubble generating device 10, more specifically from a liquid outlet 34, namely from an outlet opening of the casing 16, into the nano-bubble generating device 10 through the fluid-introducing tube 20 so that the nano-bubbles are generated through the gas-permeable member 14 and contained in the nano-bubble-containing liquid or gel composition, whereby the liquid or gel composition having a desired concentration of the nano-bubbles can be obtained, so that the pharmaceutical composition containing the predetermined size of the nano-bubbles within the preferable range of concentration can be advantageously obtained.

The drug used in the present invention is selected from known drugs according to purposes and is used in the form of a liquid or a gel. Especially, the pharmaceutical composition according to the present invention is advantageously applied for dental therapy. The drug is induced toward the affected region by the advantageous effect of accelerated penetration of the pharmaceutical composition achieved by the nano-bubbles contained therein, so that the intended drug effectively penetrates into the target penetration region affected by tooth decay and the like.

Described more specifically, with respect to a pharmaceutical composition for dental therapy used in the case of damage or partial loss of the dental pulp, which is administered in the case of the damage or partial loss of the dental pulp and which permits formation of the dental pulp and/or dentin by causing differentiation of an odontoblast from a dental pulp cell in the portion to which the pharmaceutical composition is administered, examples of the drug include ones each containing as an effective ingredient at least one of MMPs (Matrix Metalloproteinase), BMPs (Bone Morphogenetic Protein), bFGF, G-CSF, CXCL14, MCP1, SDF-1, PDGF, GM-CSF, HGF, BDNF and NPY.

With respect to a pharmaceutical composition for dental therapy for treating tooth decay, which promotes sterilization, anti-inflammation and analgesia, or regeneration of the dentin, dental pulp or periodontal tissue, at least one drug is selected from: sodium hypochlorite; hydrogen peroxide; formalin cresol; formalin guaiacol; phenol; phenol camphor; parachlorophenol camphor; cresatin; guaiacol; cresol; iodine tincture; an EDTA product; calcium hydroxide; tetracycline hydrochloride; ampicillin; imipenem; panipenem; vancomycin; chloramphenicol; PBSS; PBSC; ofloxacin; levofloxacin; metronidazole; cefaclor; ciprofloxacin; minocycline; imidazole; a cathepsin K inhibitor; BMPs; bFGF; G-CSF; CXCL14; MCP1; SDF-1; PDGF; GM-CSF; HGF; BDNF and NPY. Meanwhile, drugs used for the root canal or dental caries therapy may include, in addition to the above-indicated drugs: sodium fluoride; sodium fluorophosphate; stannous fluoride; an acidic phosphate fluoride solution (APF); xylitol; POs-Ca (phosphorylated oligosaccharide calcium) and hydroxyapatite.

With respect to a pharmaceutical composition for periodontal disease, which promotes sterilization, anti-inflammation and analgesia, or regeneration of the periodontal tissue, at least one drug is selected from: iodine tincture; an EDTA product; calcium hydroxide; tetracycline hydrochloride; ampicillin; imipenem; panipenem; vancomycin; chloramphenicol; PBSS; PBSC; ofloxacin; levofloxacin; metronidazole; cefaclor; ciprofloxacin; minocycline; imidazole; a cathepsin K inhibitor; BMPs; bFGF; G-CSF; CXCL14; MCP1; SDF-1; PDGF; GM-CSF; HGF; BDNF; NPY and Emdogain®.

Furthermore, with respect to a pharmaceutical composition for dental therapy for treating alveolar pyorrhea, which cures hyperesthesia, at least one drug is selected from: potassium nitrate; oxalic acid; a diamine silver fluoride product; copal resin; sodium fluoride; zinc chloride; a water-soluble aluminium compound; a water-soluble calcium compound; BMPs and bFGF.

In addition, with respect to a pharmaceutical composition for dental therapy used in oral and implant care, which promotes sterilization, anti-inflammation and analgesia, recalcification of tooth, or regeneration of the periodontal tissue, at least one drug is selected from: benzalkonium; chlorhexidine gluconate; sodium N-lauroylsarcosinate; isopropylmethyl phenol; ε-aminocaproic acid; sodium hypochlorite; hydrogen peroxide; formalin cresol; formalin guaiacol; phenol; phenol camphor; parachlorophenol camphor; cresatin; guaiacol; cresol; iodine tincture; an EDTA product; calcium hydroxide; tetracycline hydrochloride; ampicillin; imipenem; panipenem; vancomycin; chloramphenicol; PBSS; PBSC; ofloxacin; levofloxacin; metronidazole; cefaclor; ciprofloxacin; minocycline; imidazole; a cathepsin K inhibitor; BMPs; bFGF; G-CSF; CXCL14; MCP1; SDF-1; PDGF; GM-CSF; HGF; BDNF and NPY, for example.

Further, at least one drug for use for periodontal disease therapy is selected from: isopropylmethyl phenol; thymol; clove oil; dipotassium glycyrrhizinate; allantoin; hinokitiol; cetylpyridinium chloride; panthenol; tocopherol acetate; sodium lauroyl sarcosine; tranexamic acid; ε-aminocaproic acid; bisphosphonate; tetracycline; presteron; minocycline; doxycycline; ofloxacin; levofloxacin; metronidazole; amoxicillin; a cathepsin K inhibitor; chlorhexidine; hypochlorous acid; BMPs; bFGF and Shoyo (a product of Kobayashi Pharmaceutical Co., Ltd., Japan).

The pharmaceutical composition containing the nano-bubbles according to the present invention may be applied for animals as well as the humans. In particular, the pharmaceutical composition is used in the field of dental or medical therapy via a mucous membrane or a skin, for the purposes of sterilization, disinfection, anti-inflammation and analgesia, protection of a mucous membrane and a skin, and the like. Particular application methods of the pharmaceutical composition include, with respect to the dental therapy, for example, use of devices such as a syringe for the dental therapy, a plate for the dental therapy and a nebulizer so as to introduce the intended drug into the target region, thereby permitting prevention of dental diseases, curing of dental caries, periodontal diseases, hyperesthesia and the like, and further regeneration of the dental tissue.

Described more specifically, as the syringe for the dental therapy used in the above-described application method, one of the following is selected: a syringe for introduction of the drug into the root canal, which is inserted into the root canal and introduces the drug into the root canal; a syringe for caring the dental caries used for introducing the drug into the region affected by the dental caries; a syringe for caring the periodontal diseases used for introducing the drug into the region affected by the periodontal disease; a syringe for caring the hyperesthesia used for introducing the drug into the region affected by the hyperesthesia, and the like. Meanwhile, as the plate for the dental therapy used in the above-described application method, one of the following is selected: a plate for oral care used for introducing the drug to the teeth in the oral cavity or the entirety of the periodontal tissue; a plate for caring an affected tooth, which is used for partially introducing the drug to the portions affected by the hyperesthesia or periodontitis around the implant, and the like.

Introduction of the drug into the target region by the nebulizer is performed such that the pharmaceutical composition containing the nano-bubbles in the form of a mist is absorbed into a fibrous absorbent such as a non-woven fabric, a woven fabric, a fabric or a Japanese paper, and applying it to the affected region, so that effective penetration of the drug into the affected region is achieved. The absorbent is attached to the affected region according to conventional methods. It goes without saying that the pharmaceutical composition containing the nano-bubbles and in the form of a mist can be sprayed directly to the affected region without using the fibrous absorbent such as the non-woven fabric, the woven fabric, the fabric or the Japanese paper, thereby permitting immediate penetration of the drug.

Described in more detail, the pharmaceutical composition in the form of a liquid or a gel containing nano-sized minute bubbles is introduced into the apical area or lateral branch side of the root canal by using the syringe for introduction of the drug into the root canal, and the pharmaceutical composition described above is introduced into the periodontal pocket by using a syringe for caring the periodontal diseases. Furthermore, the pharmaceutical composition is introduced into a defective part of the dental enamel by using the syringe for caring the hyperesthesia.

The target region of introduction of the drug performed by the above-described syringe for the dental therapy include, for example, at least one of the periapical lesion of the apical area, the root canal (including a blocked or curved root canal), the accessory canal, the lateral branch, the dentin tubule, the periodontal pocket and a defective part of the dental enamel.

The pharmaceutical composition containing the nano-bubbles according to the present invention may be applied to the affected region as an administrative agent in the form of a capsule comprising the pharmaceutical composition within its soluble outer shell, as known in the art. In addition, the pharmaceutical composition may be sprayed and subjected to absorption into the fibrous absorbent such as the non-woven fabric, the woven fabric, the fabric or the Japanese paper, and applied to the affected region. Furthermore, the pharmaceutical composition may be applied by directly spraying it to the affected region. Application of the pharmaceutical composition according to the present invention by spraying in the form of a mist permits prevention of the infectious disease in general.

In particular, the pharmaceutical composition containing the nano-bubbles according to the present invention exhibits an advantage with respect to a sterilization treatment of the target region. Such target region is not particularly limited, and a tooth or a periodontal tissue is advantageously selected as the target region. Especially, the pharmaceutical composition is effectively used for caring the periapical lesion of the apical area, the root canal (including the blocked or curved root canal), the accessory canal, the lateral branch, the dentin tubule, the periodontal pocket and a defective part of the dental enamel.

Meanwhile, the drug used in the above-described treatment is not particularly limited as far as it is capable of disinfection or sterilization of the target region, or capable of enlarging and cleaning of the root canal. Examples of such drug include: a solution of sodium hypochlorite; a solution of hydrogen peroxide; a formalin product (such as formalin cresol and formalin guaiacol); a phenol product (such as phenol, phenol camphor, parachlorophenol camphor, cresatin, guaiacol and cresol); an iodine product (iodine tincture); a solution of calcium hydroxide and an EDTA product (such as Smearclean®, which is a 3% EDTA solution, and MORHONINE®, which is an edetate disodium).

Also an antibacterial agent, antibiotics or a factor for cell proliferation/differentiation can be selected as the drug. For example, the following can be used: tetracycline hydrochloride; ampicillin; imipenem; panipenem; vancomycin; chloramphenicol; PBSS; PBSC (penicillin for gram-positive bacteria, bacitracin for penicillin-resistant strain, Streptomycin for gram-negative bacteria and sodium caprylate for yeast); ofloxacin; levofloxacin; metronidazole; cefaclor; ciprofloxacin; imidazole; a cathepsin K inhibitor; BMPs; bFGF; and the like.

### Examples

To clarify the present invention more specifically, some examples of the present invention will be described. However, it is to be understood that the present invention is by no means limited by the details of the illustrated examples and that the invention may be embodied with various changes, modifications and improvements not described below in the examples, and above in the detailed description, which may occur to those skilled in the art without departing from the spirit and scope of the invention.

[Experiment 1]

—In Vitro Sterilization Test of the Root Canal Using an Extracted Canine Front Tooth—

The root canal of an extracted canine front tooth was subjected to enlargement by removal of the root canal wall using a reamer #60, and subjected to removal of a smear layer with an EDTA product (Smearclean®: a product of Nippon Shika Yakuhin Co., Ltd., Japan). Then, the inside of the root canal was dried with a cotton plug, and the apical hole was filled with a self-curing resin. Subsequently, a kanamycin-resistant E. faecalis labeled by Green Fluorescence Protein (GFP) cultured within a liquid culture medium of Brain Heart Infusion (BHI) was injected into the root canal, and the root canal was temporarily sealed with an impression material and a film. Then, the kanamycin-resistant E. faecalis was subjected to an aerobic culture at a temperature of 37° C. in a humid environment for seven days, such that a deep part of the root canal was infected.

Meanwhile, 2 L of distilled water as a fluid was introduced under a predetermined pressure through the fluid-introducing tube (20) of the nano-bubble generating device (10) shown in FIG. 1 so as to flow through the fluid passage (26), while a compressed air as a gas was introduced through the gas-introducing tube (18) and ejected from the crazed film (30) placed on the outer surface of the gas-permeable member (14). Thus, a distilled water containing minute bubbles was prepared. Further, the distilled water containing the minute bubbles, which flows from the liquid outlet (34) of the nano-bubble generating device (10), was reintroduced into the nano-bubble generating device (10) through the fluid-introducing tube (20) so that the distilled water containing the minute bubbles was subjected to circulation in the nano-bubble generating device (10). This operation was performed repeatedly so as to increase the concentration of the minute bubbles (nano-bubbles) within the distilled water. Then, the distilled water was subjected to circulation for 5 minutes and a nano-bubble water containing the minute bubbles (nano-bubbles) having a bubble diameter of 40 nm-400 nm (an average bubble diameter: 114 nm, D₅₀: 91 nm) and a concentration of 6.8×10⁷ bubbles/ml was obtained. It is noted that the bubble diameter and the concentration of the nano-bubbles within the nano-bubble water was measured by using a nano-particle analyzer (NanoSight LM-20) available from Quantum Design Japan, Inc.

Subsequently, the temporary sealing material was removed from the above-described infected root canal, and the root canal was irrigated with 5 mL of the saline. Then, the inside of the root canal was dried by using a sterilized paper point, and subjected to treatments under the following conditions A)-E). It is noted that the nano-bubble water was used in the following treatments after it was subjected to filtration and sterilization.

A) An experimental control (no injection into the root canal)

B) 20 μL of a pharmaceutical composition having an ampicillin concentration of 10 mg/mL and obtained by mixing 50 parts by volume of the nano-bubble water and 50 parts by volume of an ampicillin solution was injected into the root canal, and the root canal was left for 5 minutes.

C) 20 μL of a pharmaceutical composition having an ampicillin concentration of 10 mg/mL and obtained by mixing 50 parts by volume of the nano-bubble water and 50 parts by volume of an ampicillin solution was injected into the root canal, and the root canal was left for 10 minutes.

D) 20 μL of a pharmaceutical composition having an ampicillin concentration of 10 mg/mL and obtained by mixing 99 parts by volume of the nano-bubble water and 1 part by volume of an ampicillin solution was injected into the root canal, and the root canal was left for 5 minutes.

E) 20 μL of a pharmaceutical composition having an ampicillin concentration of 10 mg/mL and obtained by mixing 99 parts by volume of the nano-bubble water and 1 part by volume of an ampicillin solution was injected into the root canal, and the root canal was left for 10 minutes.

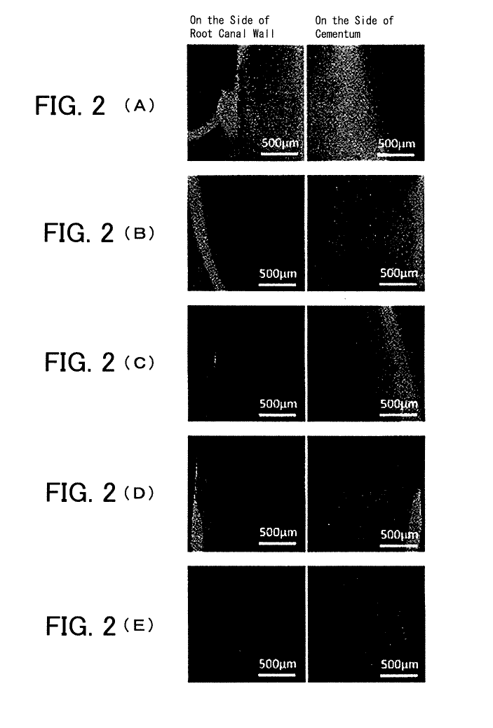

For confirming the amount of the bacteria existing within the root canal, each of the root canals subjected to the above treatments was cleaned with the sterilized saline with respect to its inside and dried by using the sterilized paper point. Then, the root canal was subjected to injection of the saline and sliced to a thickness of about 300 μm after two days of the aerobic culture of the bacteria under a humid environment. The sliced root canal was observed through a confocal laser microscope to measure a range within which GFP-labeled fluorescence-emitting E. faecalis was observed, and an effect of mixing of the nano-bubble water was evaluated. FIGS. 2 (A)-(E) represent the observation images through the confocal laser microscope corresponding to the above-described respective treatments A)-E).

As is apparent from the observation images shown in FIG. 2, with respect to the control group treated by the treatment A), it is recognized that the entirety of the root canal wall generates fluorescence (see FIG. 2 (A)). On the other hand, with respect to the treatment B) wherein the root canal was subjected to injection of the pharmaceutical composition containing the same amounts of the nano-bubble water and the ampicillin solution and was left for 5 minutes, the root canal generates fluorescence in its deep part not less than 600-700 μm from the root canal wall (see FIG. 2 (B)). With respect to the treatment C) wherein the root canal was subjected to injection of the above-described pharmaceutical composition and was left for 10 minutes, the root canal generates fluorescence in its deep part not less than 900-1000 μm from the root canal wall, as shown in FIG. 2(C). Furthermore, with respect to the treatment D) wherein the root canal was subjected to injection of the pharmaceutical composition containing the larger amount of the nano-bubble water than that of the ampicillin solution and was left for 5 minutes, the root canal generates fluorescence in its deep part not less than 1000-1300 μm from the root canal wall, as shown in FIG. 2(D). With respect to the treatment E) wherein the root canal was left for a longer time after injection, the root canal generates fluorescence in its deep part not less than 1000-1500 μm from the root canal wall, as shown in FIG. 2(E). According to the above-described results, it is recognized that a larger proportion of the nano-bubble water mixed in the pharmaceutical composition, namely a larger amount of mixing of the nano-bubbles, and a longer time wherein the root canal was left after injection of the pharmaceutical composition, permit a larger area of sterilization of the root canal. Thus, it is confirmed that the nano-bubbles permit the ampicillin to penetrate into and function in the deeper part of the root canal wall.

[Experiment 2]

—In Vivo Sterilization Test of a Tooth Having an Infected Root Canal Using Nano-Bubbles and an Antibacterial Drug—

A front tooth of a dog under general anesthesia was subjected to opening of the pulp chamber according to the conventional method, and its root canal was enlarged with the reamer #60. The root canal was left for 15 days in an open state with a pledget being placed on its orifice, so that an artificial infected root canal was prepared. After the first extraction of bacteria was performed to confirm the number of bacteria before the operation, the infected root canal was repeatedly irrigated by using 2 ml of a 3-5% solution of sodium hypochlorite and 2 ml of a 3% solution of hydrogen peroxide in turn, and further irrigated with saline. Subsequently, the inside of the root canal was dried by using the sterilized paper point. The dried root canal was subjected to injection of a solution of Vibramycin® wherein Vibramycin® was added to the nano-bubble water prepared in the above Experiment 1 in a concentration of 35 μg/mL, and was left for 5 minutes. Afterward, the root canal was subjected to irrigation with saline, medical application of the above-described solution of Vibramycin® having the concentration of 35 μg/mL by the paper point, and temporary sealing with a filling (stopping) and a composite resin. After removal of the temporary sealing and the second extraction of bacteria performed one week after the medical application, the operation consisting of the following steps was performed in the same manner as described above: the irrigation by using the solution of sodium hypochlorite and the solution of hydrogen peroxide in turn; the injection of the above solution of Vibramycin® and subsequent leaving for 5 minutes; and the medical application of the solution of Vibramycin® using the paper point and the temporary sealing. Subsequently, one week after the above operation, the third extraction of bacteria was performed, followed by the operation described above. Further subsequently, the fourth extraction of bacteria was performed, followed by the operation described above. Then, one week after the fourth operation, the fifth extraction of bacteria was performed, in advance of a stem cell transplantation of the dental pulp.

On the other hand, as a control group, the first extraction through the fifth extraction of bacteria were performed as described above by using an antibacterial drug solution (without nano-bubbles) wherein the concentration of Vibramycin® was adjusted to 35 μg/ml with saline, in place of the above-described solution of Vibramycin®.

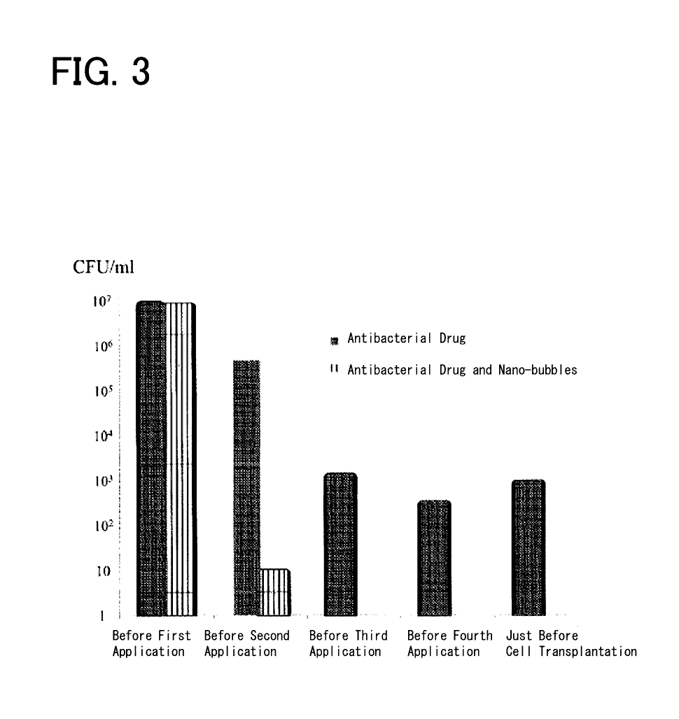

Each of the samples subjected to the extraction of bacteria was inoculated to a blood agar based on a limiting dilution method, and the number of bacteria was counted after 5 days of anaerobiotic culture. The results are shown in FIG. 3 in contrast to the cases wherein only the antibacterial drug was used (the control group). It is noted that a statistical treatment was performed with a non-parametric test.

As is apparent from FIG. 3, with respect to the root canal treated by the solution of Vibramycin® wherein the nano-bubbles were introduced according to the present invention, the number of bacteria became undetectable in the third extraction of bacteria, proving that the nano-bubbles have an advantageous effect of the accelerated penetration of the drug into the affected region.

[Experiment 3]

—In Vivo Regeneration of the Dental Pulp and Periapical Tissue of a Tooth Having an Infected Root Canal Using Nano-Bubbles and an Antibacterial Drug—

The inside of the infected root canal of a tooth subjected to the operation described in the Experiment 2 was irrigated by using 2 ml of a 3-5% solution of sodium hypochlorite and 2 ml of a 3% solution of hydrogen peroxide in turn. Then, the root canal was irrigated with 5 mL of saline, exposed to an EDTA product (Smearclean®) for 60 seconds, further irrigated with saline, and dried by using the sterilized paper point.

Meanwhile, 5×10⁵ autologous stem cells of dental pulp, which were collected by an induced mobilization method and frozen after 6 generations of subculturing, were suspended in 20 μL of Atelocollagen Implant (a product of Koken Co., Ltd., Japan), and further 1.5 μL of a 100 μg/mL solution of Granulocyte-Colony Stimulating Factor (G-CSF) was suspended therein, so that the obtained suspension was injected into the root canal. Then, a gelatin sponge for hemostasis (Spongel®: a product of Astellas Pharma Inc., Japan) was put onto the root canal, and its cavity was sealed with a glass ionomer cement (FUJI IX: a product of GC Corporation, Japan) and a composite resin (CLEARFIL MAJESTY LV: a product of Kuraray Noritake Dental Inc., Japan).

14 days after the transplantation of the above stem cells of the dental pulp, the tooth including its periapical tissue was removed from the dog. Then, a paraffin section of a thickness of about 5 μm in its vertical section was prepared from the tooth according to the conventional method, which paraffin section was subjected to Hematoxylin-Eosin staining (HE staining), so as to be subjected to observation of its form to confirm whether the dental pulp was formed or not. Immunostaining operations were performed by lectin with respect to angiogenesis, and by PGP 9.5 with respect to neurite outgrowth. In addition, differentiation of an odontoblast-like cell adhering to the side wall was examined by in situ hybridization of Dentin Sialophosphoprotein (DSPP) and Enamelysin.

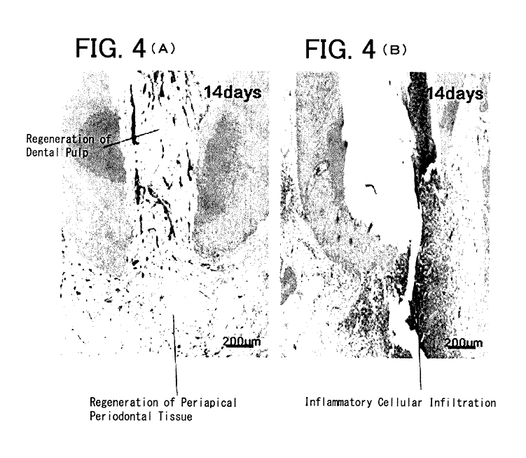

As shown in FIG. 4(A), regeneration of the dental pulp tissue and the periapical tissue was observed after 14 days, while inflammatory cellular infiltration and internal absorption were hardly observed. On the wall of the dentin, an odontoblast extending its process to the dentin tubules and appearance of an mRNA of Dentin Sialophosphoprotein (DSPP) and Enamelysin, which are the marker of the odontoblast, were observed. Furthermore, the angiogenesis and the neurite outgrowth were observed within the regenerated dental pulp tissue.

On the other hand, as shown in FIG. 4(B), in the case of a similar transplantation only with the medical application, alveolar bone resorption and inflammatory cellular infiltration were observed, while regeneration of the dental pulp tissue was hardly observed.

According to the above results, it is recognized that mixing of the nano-bubbles into the pharmaceutical composition permits a dramatical effect with respect to the regeneration of the dental pulp, dentin or periodontal tissue within the tooth having the infected root canal.

[Experiment 4]

—In Vivo Decrease of the Number of Bacteria within a Periodontal Pocket by Using Both of Nano-Bubbles and an Antibacterial Disinfecting Drug—

An affected tooth (maxillary) of a research subject was subjected to removal of dental plaque with a pledget, and to extraction of bacteria by insertion of a #25 paper point into each of 5 points in the periodontal pocket on its buccal side. The extracted bacteria was put into a special cup (DU-AC-02NP-H: a product of Panasonic Corporation, Japan) and subjected to measurement with respect to its number by using a bacterial counter (DU-AC-01NP-H: a product of Panasonic Corporation, Japan).

5 drops of ConCool (a 0.05% solution of chlorhexidine gluconate: a product of Weltec Corporation, Japan) were added to 25 mL of sterilized distilled water, and further a sterilized gel (Ultrasound transmission gel, Aquasonic 100: Parker Lab. Inc., U.S.A.) was added at a ratio of 1/25, so that a ConCool dilution was prepared. The dilution was injected into the dental pocket on the buccal side of the affected tooth. After one minute, the research subject was made to gargle, and the affected tooth was dried by air. Then, the extraction of bacteria was performed at 5 points within the periodontal pocket on the buccal side to count the number of bacteria, as described above.

Furthermore, a ConCool dilution having the same concentration as the above (the 1/25 concentration of the gel) was prepared so as to contain the nano-bubble water prepared by the method as in the case of Experiment 1 at a ratio of 50 volume % and injected into the periodontal pocket on the buccal side of the affected tooth by using a tip. After one minute, the research subject was made to gargle, and the affected tooth was dried by air. Then the extraction of bacteria was performed at 5 points within the periodontal pocket on the buccal side to count the number of bacteria, as described above.

As a result of the above operations, the number of bacteria, which had been 1.0×10⁷ CFU/mL before the operation, was reduced to approximately 1/3, namely 3×10⁶ CFU/mL, by using the ConCool dilution without the nano-bubbles. Furthermore, it was recognized that the ConCool dilution containing the nano-bubbles permitted dramatic reduction of the number of bacteria to an undetectable degree, proving an advantageous effect of the nano-bubbles to reduce the number of bacteria within the periodontal pocket with respect to the periodontal diseases.

[Experiment 5]

—In Vivo Decrease of the Number of Bacteria within the Periodontal Pocket by Applying Nano-Bubbles and an Antibacterial Disinfecting Drug by Using a Plate—

First, a plate (mold) for covering the entirety of the maxillary premolar portion of a dog was made from a silicone impression material in the form of a paste. Each of the second and third maxillary premolars of the dog was subjected to removal of dental plaque with a pledget, and to extraction of bacteria by using a #25 paper point from each of 2 points in the periodontal pocket on its buccal side. The sample of bacteria was preserved in a sterile and anaerobic state within a solution of PLADIA (available from Showa Yakuhin Kako Co., Ltd., Japan) as a transport solution.

Subsequently, 5 drops of ConCool (a 0.05% solution of chlorhexidine gluconate: a product of Weltec Corporation) were added to 25 mL of sterilized distilled water, and further a sterilized gel (Ultrasound transmission gel, Aquasonic 100: Parker Lab. Inc., U.S.A.) was added at a ratio of 1/10, so that a ConCool dilution (a gel solution) was prepared. After application of this gel solution to the part of the plate corresponding to the second maxillary premolar, the plate was put on the dog to cover the entirety of its maxillary premolar portion, and the second maxillary premolar was subjected to the effect of the gel solution for 5 minutes. Then, the second maxillary premolar was irrigated by the sterilized saline and dried by air. Then, the extraction of bacteria was performed at 2 points within the periodontal pocket on the buccal side. A sample of the extracted bacteria was preserved within a PLADIA solution.

Furthermore, a ConCool dilution having the same concentration as the above (the 1/10 concentration of the gel) was prepared so as to contain the nano-bubble water prepared by the method as in the case of Experiment 1 and was further subjected to filtration and sterilization by a filter. After application of this gel solution to the part of the plate corresponding to the third maxillary premolar, the plate was put on the dog to cover the entirety of its maxillary premolar portion, and the third maxillary premolar was subjected to the effect of the gel solution for 5 minutes. As in the case of the above, the third maxillary premolar was then irrigated by the sterilized saline and dried by air. Then the extraction of bacteria was performed at 2 points within the periodontal pocket on the buccal side. A sample of the extracted bacteria was preserved within a PLADIA solution.

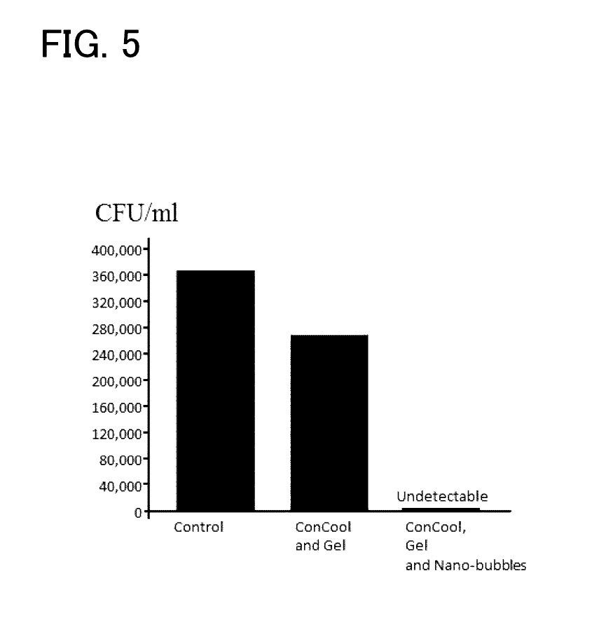

Each of three samples subjected to the extraction of bacteria and preserved as described above was inoculated to a blood agar based on a multiple-dilution method, and the number of colonies was counted after 5 days of anaerobiotic culture. A statistical treatment was performed with a non-parametric test. The results are shown in FIG. 5.

As is apparent from the results shown in FIG. 5, the number of bacteria, which had been 3.7×10⁵ CFU/mL before the operation, was reduced to about 2.7×10⁵ CFU/mL, by using the ConCool dilution without the nano-bubbles. Furthermore, it was recognized that the ConCool dilution containing the nano-bubbles permitted dramatic reduction of the number of bacteria to an undetectable degree. The results show that mixing of the nano-bubble water can remarkably improve a degree of reduction of the number of bacteria within the periodontal pocket with respect to the periodontal diseases.

[Experiment 6]

—In Vivo Verification of Safety of Nano-Bubbles—

A front tooth of a dog under general anesthesia was subjected to opening of the pulp chamber according to the conventional method, and its root canal was enlarged with a reamer #45. Then, the root canal was repeatedly irrigated by using a 3-5% solution of sodium hypochlorite and a 3% solution of hydrogen peroxide in turn, and further irrigated with saline. Subsequently, the inside of the root canal was dried by using the sterilized paper point. The dried root canal was subjected to injection of the nano-bubble water, and was left for 5 minutes. After further irrigation with saline, the root canal was temporarily sealed by using a glass ionomer cement (FUJI IX: a product of GC Corporation, Japan) and a composite resin (CLEARFIL MAJESTY LV: a product of Kuraray Noritake Dental Inc., Japan). After one week and after two weeks, the temporary sealing was removed and the irrigation was performed by using the solution of sodium hypochlorite and the solution of hydrogen peroxide in turn. Furthermore, the nano-bubble water was injected as in the case of the first operation.

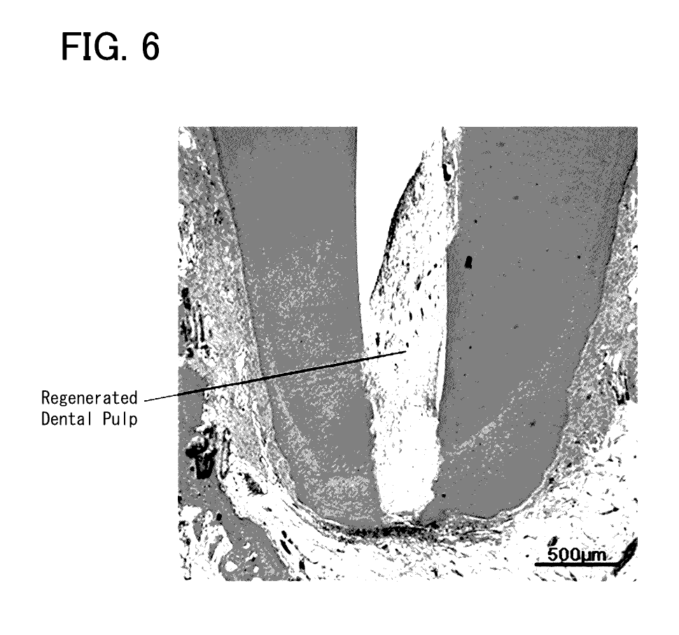

Three weeks after the first injection of the nano-bubble water, the tooth including its periapical tissue was removed from the dog. Then, a paraffin section of a thickness of about 5 μm in its vertical section was prepared from the tooth according to the conventional method, which paraffin section was subjected to HE staining, so as to be subjected to observation of its form. In addition, every one week after the first injection of the nano-bubble water, a blood sample was taken from the dog in order to evaluate toxicity of the nano-bubble water. As a result, as shown in FIG. 6, any inflammatory cell infiltration or internal absorption was not observed around the root canal, proving the safety of the nano-bubble water. Furthermore, also with respect to the blood test, no harmful influence such as toxicity to the entire body due to the nano-bubble water was observed, so that the safety of the nano-bubble water was confirmed.

[Experiment 7]

—In Vivo Verification of an Effect of Accelerated Penetration of a Drug into Skin, Mucous Membrane and Gingiva Achieved by Nano-Bubbles—

With respect to a dog under general anesthesia, each of its well-dried skin, mucous membrane and gums was subjected to dripping of a drug solution containing the nano-bubble water and tetracycline (088K0680: Sigma, U.S.A.) at a ratio of 4.5 μg/mL. The nano-bubble water was prepared by the same method as in Experiment 1. Subsequently, 5 minutes after the dripping of the drug solution, the dripped drug solution was removed, and each part was collected for preparing a frozen sample in the vertical section. As an experimental control, a liquid obtained by diluting tetracycline with water was subjected to dripping as described above and frozen samples were prepared after 5 minutes as described above.

The thus obtained samples were observed through a fluorescent microscope, using a property of tetracycline to generate fluorescence by irradiation of an ultraviolet ray. As a result, it was found that the effect of penetration of the drug was increased advantageously with respect to the frozen samples subjected to the dripping of the drug solution containing the nano-bubble water, as compared with the frozen samples obtained by using the water as a diluting medium.

### INDUSTRIAL APPLICABILITY

The pharmaceutical composition capable of accelerated penetration into an affected region according to the present invention contains the nano-sized minute bubbles exhibiting an excellent effect of the accelerated penetration, and thus has a characteristic of permitting a desired drug to be directed deep into the target region such as a tooth decay, for example, and to effectively penetrate into the target region. Besides, this characteristic contributes to the penetration of the drug not only for the humans but also for animals. Furthermore, according to the present invention, the nano-sized minute bubbles may be included in various kinds of liquid (including a liquid in the form of a gel) to exhibit the effect of accelerated penetration of the drug. The effect of accelerated penetration is advantageously utilized for spray in the field of agriculture and horticulture, for surface modification of materials in various industries, and for other purposes.

### DESCRIPTION OF NUMERALS

10 nano-bubble generating device
12 base (base stand)
14 gas-permeable member
16 casing
18 gas-introducing tube
20 fluid-introducing tube
22 gas-introducing passage
24 fluid-introducing passage
26 fluid passage
28 cylindrical member
28a gas inlet hole
30 crazed film
32 binding string

1. A pharmaceutical composition in a form of a liquid or a gel capable of accelerated penetration into an affected region, comprising a predetermined drug and containing minute bubbles dispersed therein, characterized in that:
the minute bubbles are nano-bubbles each of which has a nano-meter diameter and is given a high internal pressure due to its surface tension and negatively charged.

2. The pharmaceutical composition capable of accelerated penetration into an affected region according to claim 1, wherein each of the nano-bubbles has a diameter within a range of 10 nm-1000 nm.

3. The pharmaceutical composition capable of accelerated penetration into an affected region according to claim 1, wherein the nano-bubbles are contained and dispersed at a ratio of 1×10⁶-2×10⁸ in number/ml.

4. The pharmaceutical composition capable of accelerated penetration into an affected region according to claim 1, wherein the pharmaceutical composition is a mixture obtained by mixing a solution containing the nano-bubbles, and the drug or a liquid or gel-type preliminary composition containing the drug.

5. The pharmaceutical composition capable of accelerated penetration into an affected region according to claim 1, wherein the minute bubbles having the nano-meter diameter are generated by a nano-bubble generating device comprising: a cylindrical gas-permeable portion having a gas-permeable film formed by generating crazes in a high molecular resin film arranged on its gas-permeable cylindrical outer circumferential surface, wherein a predetermined pressurized gas is ejected under the control of an amount of permeation of the gas through the gas-permeable film; gas blowing means for blowing the pressurized gas into an inside of the cylindrical gas-permeable portion; a cylindrical casing which has an inside diameter larger than the outside diameter of the cylindrical gas-permeable portion and which is open at its opposite ends; and fluid flowing means for permitting a predetermined fluid to flow through a fluid passage provided by a gap formed by accommodating the cylindrical gas-permeable portion within the cylindrical casing,
and wherein bubbles formed by the gas ejected from the gas-permeable outer circumferential surface of the gas-permeable portion are sheared and micronized by the fluid flowing through the fluid passage at an early stage of generation, whereby the minute bubbles with the nano-meter diameter are formed.

6. The pharmaceutical composition capable of accelerated penetration into an affected region according to claim 1, wherein the pharmaceutical composition is formed as an administrative agent included within a soluble outer shell of a capsule and applied to the affected region.

7. The pharmaceutical composition capable of accelerated penetration into an affected region according to claim 1, wherein the pharmaceutical composition is atomized by spraying, subjected to absorption to a fibrous absorbent such as a non-woven fabric, a woven fabric, a fabric or a Japanese paper, and applied to the affected region.

8. The pharmaceutical composition capable of accelerated penetration into an affected region according to claim 1, wherein the pharmaceutical composition is atomized by spraying and applied directly to the affected region.

9. The pharmaceutical composition capable of accelerated penetration into an affected region according to claim 1, wherein the pharmaceutical composition is intended for medical use and the drug is subjected to penetration into a deep part of the target region affected by a tooth decay and the like by utilizing an effect of accelerated penetration achieved by the nano-bubbles.

10. The pharmaceutical composition capable of accelerated penetration into an affected region according to claim 1, wherein the pharmaceutical composition is used for dental therapy in the case of a damage or a partial loss of the dental pulp, the pharmaceutical composition being administered in the case of the damage or the partial loss of the dental pulp and permitting formation of the dental pulp and/or the dentin by causing differentiation of the odontoblast from the dental pulp cell at a portion to which the pharmaceutical composition is administered, and wherein the drug includes as an effective ingredient at least one of MMPs, BMPs, bFGF, G-CSF, CXCL14, MCP1, SDF-1, PDGF, GM-CSF, HGF, BDNF and NPY.

11. The pharmaceutical composition capable of accelerated penetration into an affected region according to claim 1, wherein the pharmaceutical composition is used for dental therapy to promote sterilization, anti-inflammation and analgesia or regeneration of the dentin, the dental pulp or the periodontal tissue, the pharmaceutical composition containing as the drug at least one of sodium hypochlorite, hydrogen peroxide, formalin cresol, formalin guaiacol, phenol, phenol camphor, parachlorophenol camphor, cresatin, guaiacol, cresol, iodine tincture, an EDTA product, calcium hydroxide, tetracycline hydrochloride, ampicillin, imipenem, panipenem, vancomycin, chloramphenicol, PBSS, PBSC, ofloxacin, levofloxacin, metronidazole, cefaclor, ciprofloxacin, minocycline, imidazole, a cathepsin K inhibitor, BMPs, bFGF, G-CSF, CXCL14, MCP1, SDF-1, PDGF, GM-CSF, HGF, BDNF and NPY.

12. The pharmaceutical composition capable of accelerated penetration into an affected region according to claim 1, wherein the pharmaceutical composition is used for treating periodontal diseases in dental therapy to promote sterilization, anti-inflammation and analgesia or regeneration of the periodontal tissue, the pharmaceutical composition containing as the drug at least one of iodine tincture, an EDTA product, tetracycline hydrochloride, ampicillin, imipenem, panipenem, vancomycin, chloramphenicol, PBSS, PBSC, ofloxacin, levofloxacin, metronidazole, cefaclor, ciprofloxacin, minocycline, imidazole, a cathepsin K inhibitor, BMPs, bFGF, G-CSF, CXCL14, MCP1, SDF-1, PDGF, GM-CSF, HGF, BDNF, NPY and Emdogain®.

13. The pharmaceutical composition capable of accelerated penetration into an affected region according to claim 1, wherein the pharmaceutical composition is used for curing hyperesthesia in dental therapy, the pharmaceutical composition containing as the drug at least one of potassium nitrate, oxalic acid, a diamine silver fluoride product, copal resin, sodium fluoride, zinc chloride, a water-soluble aluminium compound, a water-soluble calcium compound, BMPs and bFGF.

14. The pharmaceutical composition capable of accelerated penetration into an affected region according to claim 1, wherein the pharmaceutical composition is used for an oral and implant care in dental therapy to promote sterilization, anti-inflammation and analgesia, recalcification of a tooth, or regeneration of the periodontal tissue, the pharmaceutical composition containing as the drug at least one of benzalkonium, chlorhexidine gluconate, sodium N-lauroylsarcosinate, isopropylmethyl phenol, ε-aminocaproic acid, sodium hypochlorite, hydrogen peroxide, formalin cresol, formalin guaiacol, phenol, phenol camphor, parachlorophenol camphor, cresatin, guaiacol, cresol, iodine tincture, an EDTA product, calcium hydroxide, tetracycline hydrochloride, ampicillin, imipenem, panipenem, vancomycin, chloramphenicol, PBSS, PBSC, ofloxacin, levofloxacin, metronidazole, cefaclor, ciprofloxacin, minocycline, imidazole, a cathepsin K inhibitor, BMPs, bFGF, G-CSF, CXCL14, MCP1, SDF-1, PDGF, GM-CSF, HGF, BDNF and NPY.

15. The pharmaceutical composition capable of accelerated penetration into an affected region according to claim 1, wherein the pharmaceutical composition is used in the field of dental or medical therapy via a mucous membrane or a skin, for the purposes of sterilization, disinfection, anti-inflammation and analgesia, protection of the mucous membrane and the skin, and for other purposes.

16. A method of producing the pharmaceutical composition capable of accelerated penetration into an affected region according to claim 1, comprising:
a step of providing a drug to be penetrated into a target affected region or a liquid or gel-type preliminary composition containing the drug;
a step of preparing a solution containing the nano-bubbles by using a nano-bubble generating device comprising: a cylindrical gas-permeable portion having a gas-permeable film formed by generating crazes in a high molecular resin film arranged on its gas-permeable cylindrical outer circumferential surface, wherein a predetermined pressurized gas is ejected under the control of an amount of permeation of the gas through the gas-permeable film; gas blowing means for blowing the pressurized gas into an inside of the cylindrical gas-permeable portion; a cylindrical casing which has an inside diameter larger than the outside diameter of the cylindrical gas-permeable portion and which is open at its opposite ends; and liquid flowing means for permitting a predetermined liquid to flow through a liquid passage provided by a gap formed by accommodating the cylindrical gas-permeable portion within the cylindrical casing, the device being characterized in that bubbles formed by the gas ejected from the gas-permeable outer circumferential surface of the gas-permeable portion are sheared and micronized by the liquid flowing through the liquid passage at an early stage of generation, whereby the minute bubbles with the nano-meter diameter are formed; and
a step of mixing the obtained solution containing the nano-bubbles, and the drug or the liquid or gel-type preliminary composition containing the drug so as to obtain the pharmaceutical composition.

17. The method of producing the pharmaceutical composition capable of accelerated penetration into an affected region according to claim 16, wherein the amount of inclusion of the nano-bubbles in a nano-bubble-containing liquid is increased by repeatedly reintroducing the nano-bubble-containing liquid flowing from an outlet opening of the casing into the nano-bubble generating device so that the nano-bubbles are further generated through the gas-permeable member in the nano-bubble-containing liquid, whereby the liquid having a desired concentration of the nano-bubbles can be obtained, so that the pharmaceutical composition containing a predetermined size of the nano-bubbles within a predetermined range of concentration can be obtained.
